(19) United States
(12) Patent Application Publication
(10) Pub. No.: US 2002/0138012 A1
(43) Pub. Date: Sep. 26, 2002

(54) Multiple parameter electrocardiograph system

(76) Inventors: Morrison Hodges, Minnetonka, MN (US); Timothy R. Church, St. Paul, MN (US)

Correspondence Address: Beck & Tysver, P.L.L.C., Suite 100, 2900 Thomas Avenue South, Minneapolis, MN, 55416-4477, US

(21) Appl. No.: 09/813,342

(22) Filed: Mar. 20, 2001

(51) Int. Cl. A61B 5/04

(52) U.S. Cl. 600/509

(57) ABSTRACT

The present invention relates to a diagnostic tool which utilizes electrocardiographic information as well as additional patient data to predict the existence of heart disease. More specifically the invention shows a model for predicting disorders treatable with implantable cardioverter defibrillators.

### FIELD OF THE INVENTION

The present invention relates generally to a diagnostic systems that use surface electrocardiograph (ECG) information, and more particularly to a multiple parameter ECG system that can be used to assist in the diagnosis of heart disease in general and, most specifically “Sudden Cardiac Death Syndrome” (SCD).

### BACKGROUND ART

The electrocardiograph (ECG) is a well-known instrument used to record the electrical activity of the heart from the body surface of the patient. In general the device is used in the clinical setting to reveal disturbances in the electrical conduction pattern of the heart. The time course of each individual heart beat gives rise to a repetitive waveform with characteristic P, Q, R, S and T segments. These electrographic manifestations of the underlying heart activity have been attributed to the propagation of the electrical activity of the atria (P wave) through the conduction system to the depolarization waveform (QRS complex) of the ventricular tissues followed by the repolarization of the ventricle which gives rise to a characteristic waveform as well (T wave). The relationship between groups of beats permits rhythm analysis where both tachyarrhythmias as well as bradyarrhythmias can be readily discerned in the ECG waveform. One may call this “rhythm analysis”.

The morphology of individual beats has also been studied with signal averaged EKGs. as well. In these studies late after potentials in the QRS complex have been identified and linked to certain incipient arrhythmias. For example, the dissociation of atria and ventricles in disorders like the Wolff-Parkinson-White syndrome can be ascertained from the ECG. This type of arrhythmia involves both atrial and ventricular chambers of the heart and both individual beats as well as beat to beat intervals can be used to diagnose the condition.

Other disorders including “sudden death” have been linked to heart rate variability, which requires a relatively long sample of heart activity. The literature includes examples of the use of T-wave alternans; QT interval duration; QT interval duration variability and/or dispersion, as well as ventricular ectopic activity for predicting sudden death. See for example U.S. Pat. No. 5,437,285 to Verrier et al. The cited reference uses a dynamic technique to monitor “alternans” in real time for use in evaluating drugs or controlling the operation of an Implantable cardioverter/defibrillator.

Although each of these measures can be extracted and computed by hand, it is now common to use computer-based rhythm analyses to improve the diagnostic value of the ECG. ECG machines can be programmed to collect and analyze data over time and they can be trained to detect certain patterns in the data. In spite of these advances it is still difficult to perform risk stratification on a patient population. Although the conventional ECG has been used alone and in combination with other data it is still difficult to effectively screen candidates for incipient “sudden death” and to identify those candidates who are suitable for placement of an implantable cardioverter-defibrillator (ICD).

Clinicians typically combine several measures of a patient's health to develop a diagnosis. For example an ECG may be used along with family history or blood chemistry to develop a diagnosis. These time-honored techniques have generated several prejudices which are addressed by this invention. For example the notion that several separate measurements from a single ECG record can have enhanced value is counterintuitive to many practitioners. Similarly many clinicians would not combine two or more measures which were ambiguous, in the hopes of refining a diagnosis. These prejudices have resulted in a “horse race” between competing measurements. Usually individual measures are compared and then one alone is selected for diagnostic use.

### SUMMARY OF THE INVENTION

This invention is disclosed in the context of the diagnosis of “sudden cardiac death” (SCD) syndrome which is an unmet need in the medical community. In operation the system divides a population of patients into a group that should receive an implantable cardioverter defibrillator (ICD) and a group that should not. The method can then be used with an individual to place them into one of the two categories. Although the invention is well suited to stratifying risk for this SCD disease it should be appreciated that the invention can be used in other disease contexts as well.

In general, the system monitors a patient and collects approximately five minutes of very high quality ECG recordings. This “single data set” or collection of electrocardiograph data is used to stratify risk of sudden death. Several independent measurements are made on the single data set. These measurements are referred to thought as “parameters”.

The parameters may be grouped into predefined categories. It is expected that a combination of the parameters selected from the categories will allow a more sensitive and selective prospective identification of those patients who will suffer from sudden death syndrome. Identification of these patients is expected to guide interventions such as the implantation of an ICD.

The preferred technique for combining the test measurements of the parameters is through the use of an “additive model” that combines both dichotomized data with continuous data. The mathematics of generalized additive models are well known in the literature. Dichotomized data has only a small number (usually two) discrete values, while the continuous data can have “any” value. It is a property of generalized additive models that they can accept and use both continuous and dichotomized data sets. The specific technique is set forth herein where each parameter is classed as continuous or dichotomized. It should be understood that as the technique is refined it made be appropriate to dichotomize some continuous measures and visa versa. Therefore it should be apparent that many variations on the disclosed technique are within the scope of the invention.

### BRIEF DESCRIPTION OF THE DRAWINGS

Though out the several views of the drawings like reference numerals refer to equivalent structure wherein:

FIG. 1 is a diagram showing the collection of ECG data;

FIG. 2 is a graphic representation of the data collected by the system:

FIG. 3 is a table categorizing the parameters;

FIG. 4 is a surface electrocardiogram representation of the computation of heart rate variability;

FIG. 5 is a surface electrocardiogram representation of the computation of ST depression;

FIG. 6 is a surface electrocardiogram representation of the computation of QT dispersion.

FIG. 7 is a surface electrocardiogram representation of the computation of heart rate variability; and,

FIG. 8 is a display of risk stratification.

### DETAILED DESCRIPTION OF THE INVENTION

Background

The purpose of this invention is to sort patients from a general population into two groups. The first group should not benefit from and therefore does not need not an ICD and the second group is likely to benefit from and therefore does require an ICD. Therefore the strategy is to stratify individuals according to their risk of having an episode of SCD in the future. This stratification is similar to both screening and to diagnosis, but with key differences. In screening, a positive screen in an otherwise healthy subject leads to the search for a definitive diagnosis of the current occult health problem through applying some “gold standard” test; here, no further diagnosis will be done and no additional projection into the future is made. Diagnosis, on the other hand, seeks to label a condition in order to guide treatment and prognosis of an existing health condition; here, no label is attached beyond the ones that led to the risk stratification in the first place. With risk stratification, based on our prediction of who will get an event and who won't, we want to separate individuals into two groups: those that need an ICD and those that don't.

Several terms will be used to distinguish related, but different concepts. A clinical measurement is any quantitative or qualitative information obtained from an individual believed to be related to that person's present health status. A predictor is any clinical measurement that is related to the probability of a future morbid event (e.g., SCD). If knowing the value of a predictor changes the probability of SCD, then the predictor is useful. A test refers to a procedure that yields either a positive or negative result, the positive result indicating a higher and a negative indicating a lower probability of a future event. Some tests come from naturally dichotomous predictors, such as the presence or absence of heart block or a specific allele. Some are dichotomizations of a single continuous predictor, such as heart rate variability or QT-interval duration. In some cases, a test can be constructed from several predictors. Answers to such questions as “Has the patient ever experienced SCD?” can be considered as “tests,” since they produce positive or negative indicators for ICD implantation.

Thorough this discussion the term “sensitivity” is the probability that a test is positive in the presence of a condition of interest; specificity is the probability that the test is negative in the absence of the condition. Thus, the ideal test has both sensitivity and specificity equal to one: it identifies all with the disease, and reassures all without it. Prevalence is the frequency of the condition among the population being tested. The positive predictive value (PPV)²⁵ of a test is simply the probability that an individual with a positive result has the disease or will experience the event of interest. It is a simple function of the sensitivity, specificity, and prevalence:

PPV={sensitivity_prevalence/[sensitivity_prevalence+(1−specificity)_(1−-prevalence)]}

The numerator is the frequency of true positives and the denominator the sum of frequencies of true positives and false positives. The negative predictive value (NPV) of a test is analogously the probability that an individual with a negative result is free of the disease, or will escape the event of interest, and also can be expressed as a function of sensitivity, specificity, and prevalence. Note that prevalence is an important component of PPV and NPV, whereas it is not a component of sensitivity and specificity.

It has been common in the past to fit a linear or at least continuous function to a complex set of estimation data. It is relatively less common to use dichotomized data with continuos data to predict censored survival times. A general discussion of additive models and there properties may be found in Generalized Additive Models by Trevor Hastie et al. Published in Statistical Science 1986, vol. 1, no. 3, at pages 297-318. Which is incorporated in its entirety herein and reproduced as an appendix text within this application.

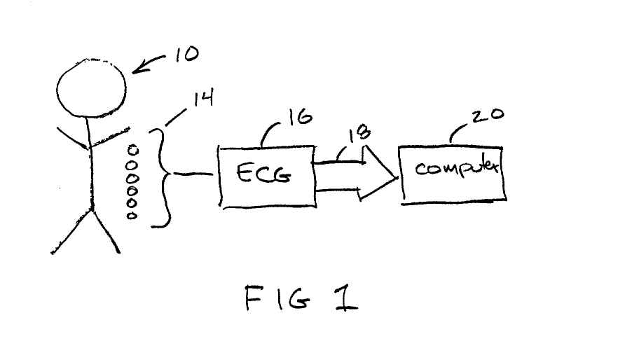

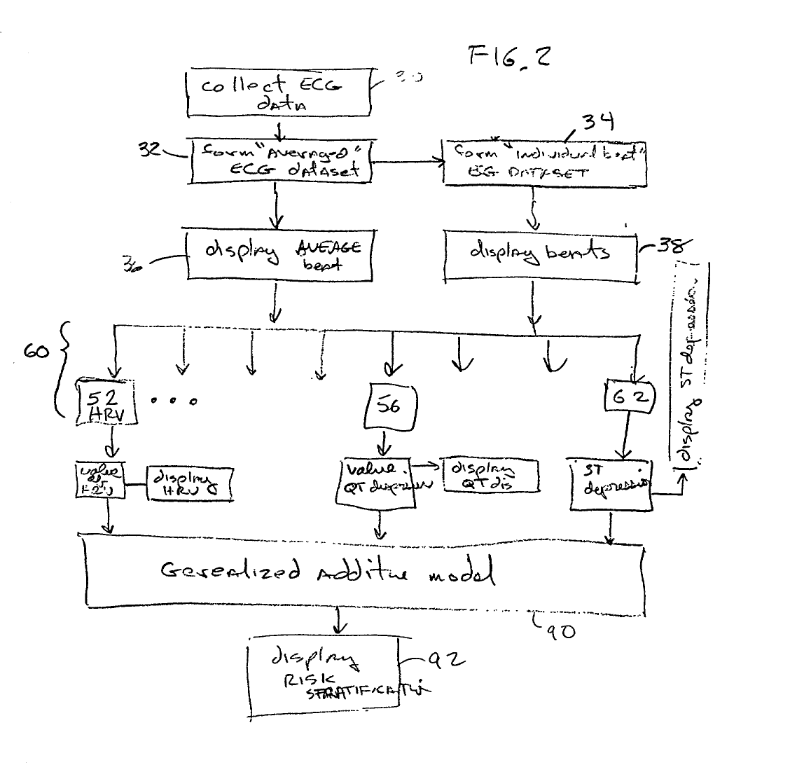

FIG. 1 shows a supine patient 10 undergoing a “resting ECG”. The patient 10 has a conventional “twelve lead” array14 of electrodes located on the chest that are connected to the ECG machine 16. The ECG machine 16 collects data for a fixed period of time on the order of five minutes. this is referred to as the single data set. The machine stores this data in a format that allows computational access to each heartbeat recorded. the raw data 18 is transferred to a computer 20 for analysis. The overall partitioning of the system is arbitrary and sufficient computing resources may exist within the ECG machine to perform the analysis.

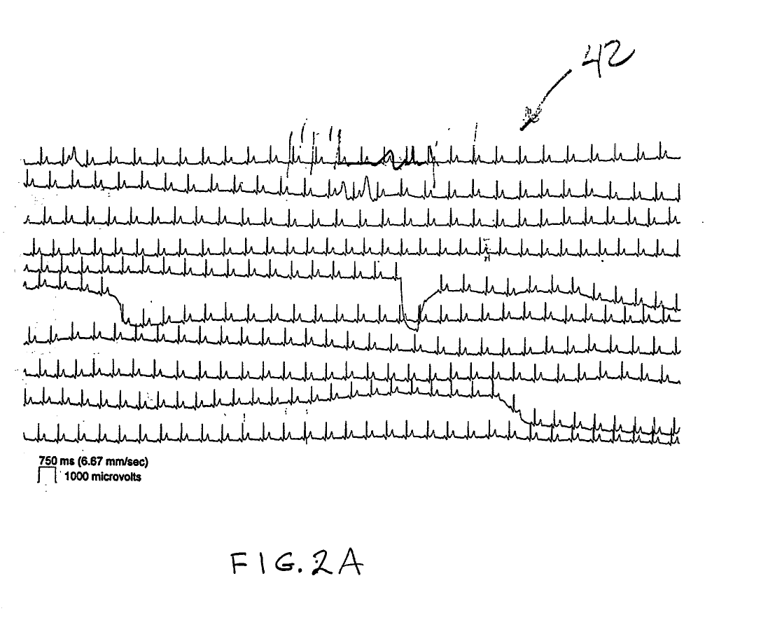

FIG. 2 shows the processes carried out in the computer 20. The raw data is collected for use in process 30. The computer system averages all normal sinus beats in process 32 and forms an averaged ECG. In process 34 the system has computational access to this averaged beat. and assess this data to define individual beta to beat intervals.

Some parameters rely on the global averaged beat computed by process 32 and some parameters rely on the individuals beats collated and collected in process 34.

Several measurements are made on these data. Individual parameters are measured and these measurements fall into broadly defined categories. FIG. 2A. is a display of data representative of a collected set of so called raw beats generally labeled 42 in the figure. This figure shows the full disclosure of all the beats collected on lead II during a sample collection window. As seen in FIG. 2A most of the experimentation presented herein has had data sets with over 300 “normal” or sinus beats taken over a five-minute interval. It is expected that the methodology will require a data set this large and that ht e data set be taken at one time important to preserve information content. The raw beats of FIG. 2A are displayed as an averaged eat 40 in FIG. 2B.

The averaging process is automated and algorithms are used to detect and select normal sinus beats and to exclude ectopic beats from the measured data. Several approaches to identify beats are known in the art. The preferred method is to exclude one interval preceding the ectopic beat and exclude the two intervals following the ectopic beat.

Although most modern ECG machines can be modified to collect the required data, the Mortara Portrait® Electrocardiograph is one device with sufficient noise performance to carry out the invention. This system has high resolution A/D conversion of 20 bits and collects 5000 samples per second per channel. The frequency is broadband and meets ANSI/AAMI standard ECIIa.

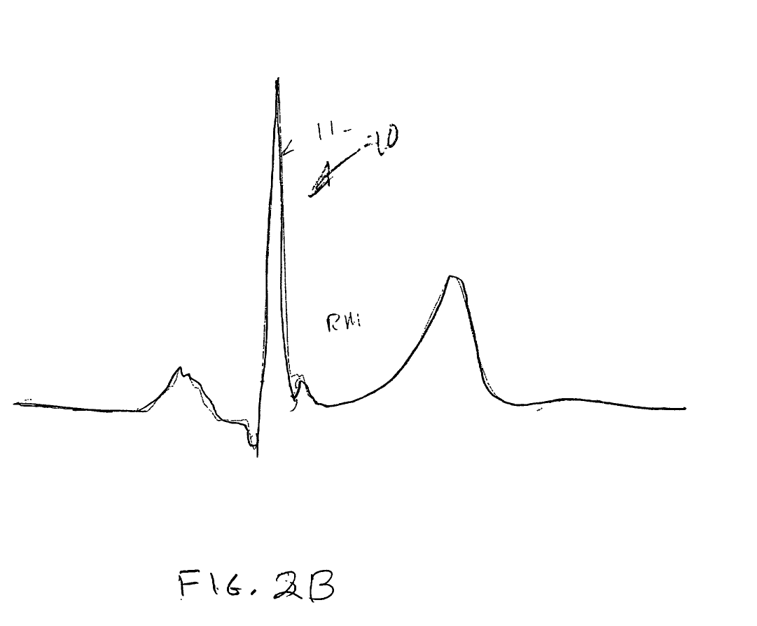

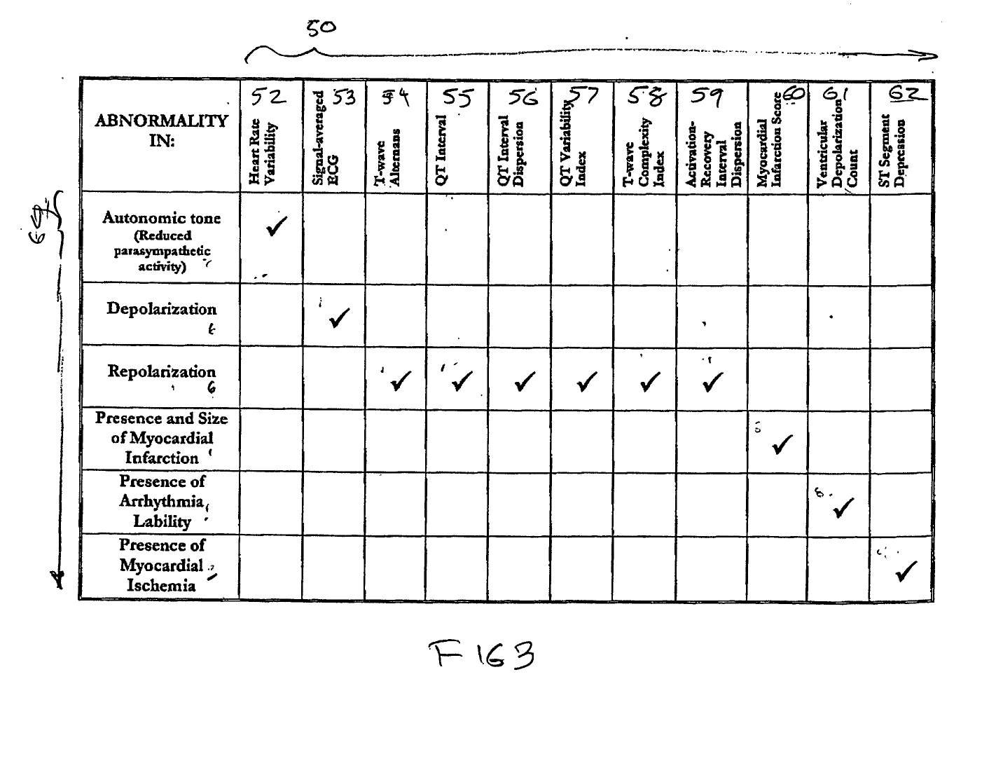

The process 36 and 38 pass data to the parameter computation s shown in process boxes labeled 52 through 62 in the FIG. 2. FIG. 3 is a table categorizing the preferred parameters shown in FIG. 2. For example the heart rate variability is computed in process 52 corresponding to parameter 52 in the table of FIG. 3. Ten representative parameter are labeled in FIG. 4 but more or less may be used in practice.

In general, measurements are made of all the parameters selected from the set of parameters disclosed. These measures are used together to stratify “sudden death” syndrome from related illnesses. It is important to note that any given parameter measurement technique can be modified without departing from the scope of the invention. The technique requires that the parameters extract information from the categories of data described in the table. These data are both “local,” in the sense that it looks to the processes occurring within one beat, and “global,” in the sense that it looks over processes that are reflected in longer intervals extending over several beats. In the table of FIG. 3, representative but not limiting parameters are enumerated along the direction 50 while the class or category of the data is set forth along direction 64. For example the check in one block indicates that the heart rate variability parameter is a measure of the autonomic tone. categorization in FIG. 3 is optional and not necessary for the additive model however it is useful for selecting proposed parameters.

Illustrative Category Descriptions

In the prior art it has been common to make several measurements and to combine these to stratify risk for subsequent SCD. However, in most instances the data are taken at various times and they do not permit multiple evaluations of a single simultaneously obtained, internally consistent data set. It has also been common to attempt to develop a single test to stratify risk in an acceptable way. In the present invention a single data set is taken over a predefined sample window. All of the tests and measures are made on this single integrated data set. However an illustrative set of six different types of measurement are made.

One measure of autonomic tone is made. Abnormalities in the autonomic nervous system are known to be a indicators of risk of SCD. The preferred ECG measurement is the variability of the heart rate. In general highly regular beat-to-beat intervals indicate risk. A measure of the whole heart depolarization process is made. The heart contracts forcefully to expel blood. The organization of this process is reflected in the surface electrogram. The preferred measure is based on the smoothness of the signal averaged electrogram in the lead II channel. For this parameter to be scored the ECG machine must have excellent noise discrimination and broadband response. This measure is easily affected by noise. A measure of the repolarization process is made. After the myocardial cells have contracted the ion pumps at the cellular level to recharge in preparation for the next beat. Abnormalities in this recharging process can lead to serious ventricular arrhythmias that are the cause of many cases of SCD. It is preferred to make a measure of the size of any infarcted myocardial tissue present in the heart. A measure of arrhythmia lability is made. The preferred technique is to count the number of ventricular ectopic beats. It is important to note that ectopic intervals and the beats themselves are excluded from the averaging process.

A measure of myocardial ischemia is made. The preferred measure is based upon ST segment depression where departures from the isoelectric potential are characterized in a continuous measurement.

Illustrative Parameter Descriptions

Block or process 52 of FIG. 2 represents a process to measure heart rate variability. In general a time measure is made between beat of the heart. It is preferred to monitor the cycle length of sequential R-waves of the heart. It is preferred to make this measure between pairs of successive “normal” R wave segments. There are numerous reports in the literature which rely on normal beats collected automatically over long periods of time using “Holter Monitors.” Although such systems are workable, it is expected that a sufficient sample size is available in five minutes of data, especially when these data are used for other parameter measurements as well. Typical units of this measure are in milliseconds (ms). The preferred technique is to measure the variance of the R-R intervals and to compute the standard deviation of the intervals. This conventional statistical technique allows computational access to a measure of the autonomic tone of the patient. The diagram of FIG. 4 is a histogram prevention of this data computation showing the number of beats at each cycle length bin. For example the largest number of beats represented by arrow 70 corresponds to a beat to beat interval of 600 ms.

Block 36 in FIG. 2 represents a process to compute the signal-averaged ECG. This process measures the morphology of the systolic action of the heart. In general the average duration of the QRS complex is taken as a measure of this parameter. The signal averaged ECG also allows the visualization of so called late potentials that are low-voltage high-frequency waveforms which are seen in patients with serious ventricular arrhythmias. This measure is made by detecting and selecting the intrinsic deflection of the heart and timing out a fixed window in time from this fiducial point. Next, a measurement window of fixed duration is established. Then the RMS voltage of the averaged beat is measured and this RMS value is used to score this parameter. The high-resolution system called for by the invention allows the detection of so-called late depolarization that reflect abnormalities in the depolarization process. The display of FIG. 2B represents this calculation for the individual beats collected in FIG. 2A by process 34 of FIG. 2. Thus FIG. 2B is the display of process 38.

The process to measure T-wave alternans may also be used as a parameter. During the course of the collection of the exercise ECG data set the patient goes from a lower metabolic activity level to a higher one. In general the measure “height” of the T wave segment of the ECG will follow a smooth and predictable course. It has been noted that a disparity in height between adjacent heartbeats is a measure of the integrity of the repolarization process. This parameter will be scored as present or absent based upon simple bands. This measurement is made on the averaged beat computed for all twelve leads. Although this is a global measurement it is expected that a subset of the twelve lead data may be appropriate for this measure. This parameter reflects an alternate measure of the cardiac repolarization process. This is a difficult measure to make and the parameter will be used only if an acceptable number of beats is collected.

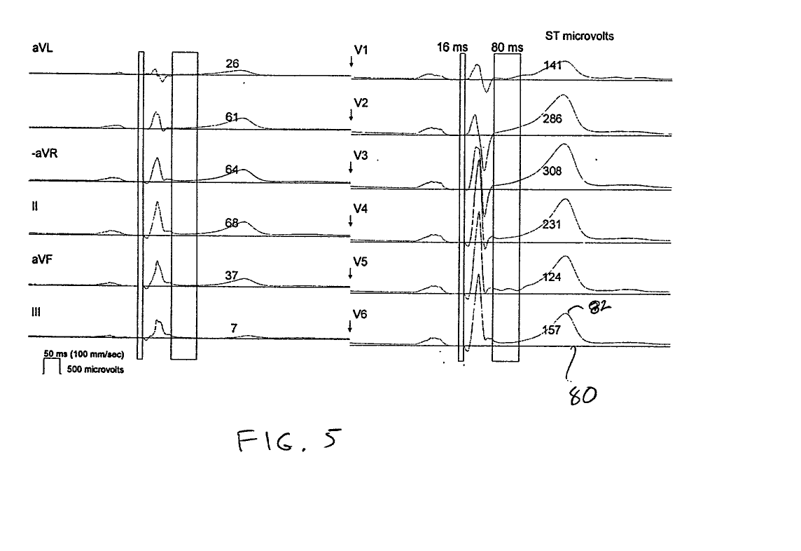

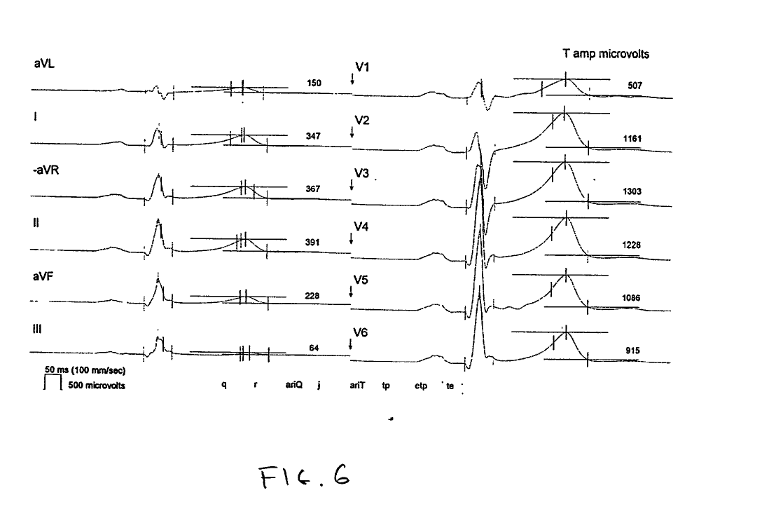

The display of FIG. 6 shows representative measures of the “height” of the QT segment used to localize the T wave measurement point. and this is an example of the output of process 56. Block 44 represents a process for measuring the QT interval dispersion. In this measure the shortest reliable measure of QT time interval is subtracted from the longest measured time interval. In general it may be useful to collect and average several short intervals and compare them to several longer intervals to stabilize the measurement. It is preferred to make this measure on all of the precordial leads and two of the limb leads. This parameter is expressed in milliseconds.

This software process for measuring QT interval variability can also establish a template and each beat-to-beat interval is compared to the template, and a score is developed which reflects how similar each beat is to the template. Several published references describe this technique. Although there is some variation in measurement technique, the measure is essentially a measure of T-wave duration and variability. Once again this parameter allows access to a measure of the global repolarization process.

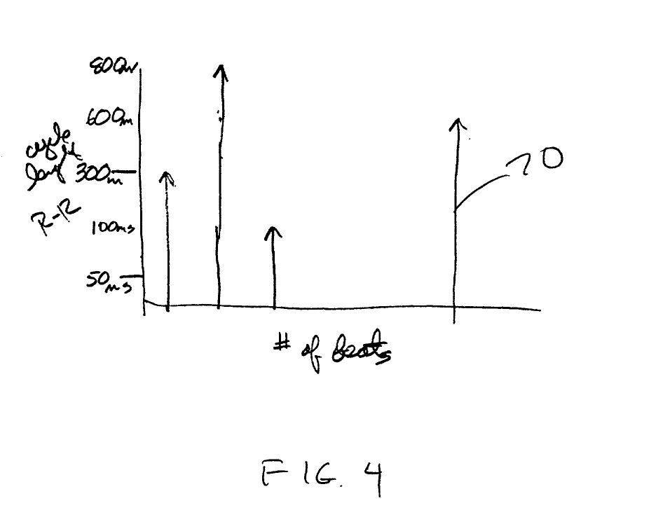

Block or process 62 represents a software process to measure ST Segment depression. This is a measure of amount of departure the ST waveform from an isoelectric potential. FIG. 5 represents this process in a graphic form and the variation of each ST segment for each beat is presented on the diagram. The process uses the averaged beat to define the isoelectric line 80 which is used as a baseline for the highest excursion of the t wave segment typified by point 82.

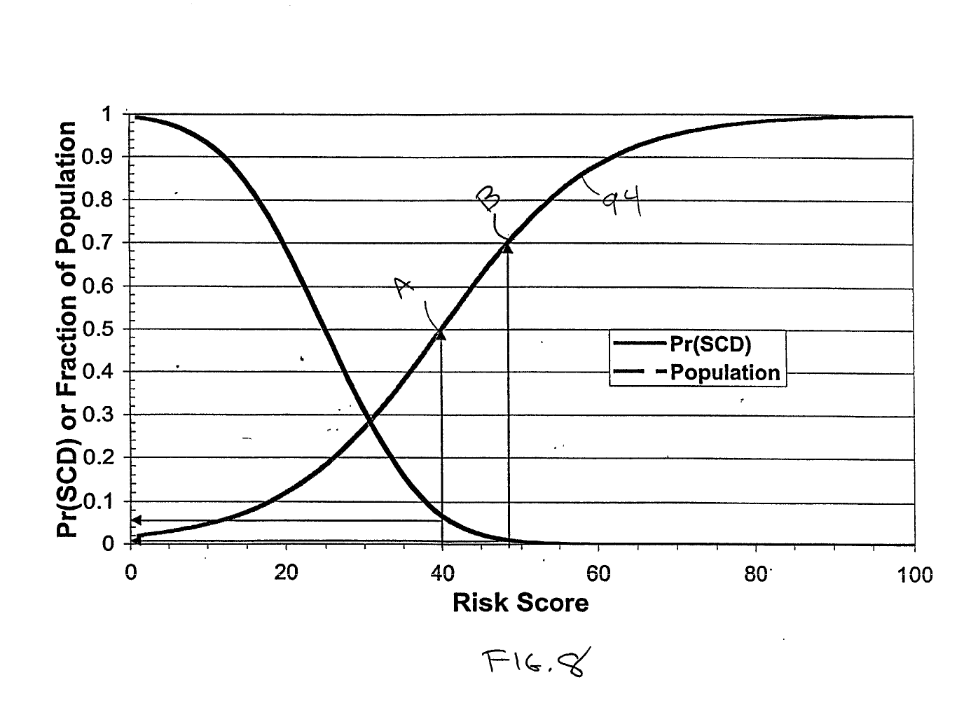

FIG. 8 represents the output of the model 90 represented in the Figure as process 92. In operation at least two parameters are submitted to the generalized additive model 90. The individual's risk is shown in the figure by curve 94. A medical judgement is made to decide whether or not to implant a device based on the score. In the Figure it may be determine that individuals with a risk between point A and point B should receive an ICD. This corresponds to a risk level between about 40 and 50.

The parameters discussed above are illustrative and preferred ways of measuring a parameter in the respective categories. However other techniques may be used as well.

What is claimed is:

1. A method of stratifying risk of sudden cardiac death (SCD) for an individual patient comprising the steps of:
a) collecting clinical data and ECG data in single recording session to compute a set of parameters, including at least two parameters selected from the group including: age,sex,ejection fraction prior MI, heart rate variability signal averaged ECG, T-wave alternans, QT interval, QT interval dispersion, QT interval variability, T-wave complexity, ST segment depression;
b) combing at least two parameters form the set of parameters in a an additive model, thereby predicting the incidence of SCD for the individual patient within a predetermined time interval.
